(19) United States
(12) Patent Application Publication
(10) Pub. No.: US 2024/0198128 A1
(43) Pub. Date: Jun. 20, 2024

(54) OPTICAL MODULATION CUFF DEVICES, SYSTEMS, AND METHODS OF MAKING AND USING

(71) Applicants: Boston Scientific Neuromodulation Corporation, Valencia, CA (US); Case Western Reserve University, Cleveland, OH (US)

(72) Inventors: Michael Jenkins, Beachwood, OH (US); Junqi Zhuo, Cleveland Heights, OH (US); Michael A. Moffitt, Solon, OH (US); Joshua Dale Howard, Sacramento, CA (US); Changfang Zhu, Valencia, CA (US)

(21) Appl. No.: 18/381,109

(22) Filed: Oct. 17, 2023

Related U.S. Application Data
(60) Provisional application No. 63/433,874, filed on Dec. 20, 2022.

(51) Int. Cl. A61N 5/06; A61N 1/05

(52) U.S. Cl. CPC A61N 5/0622; A61N 1/0556; A61N 5/0601; A61N 2005/065; A61N 2005/0665

(57) ABSTRACT

An optical lead can include a cuff body having an exterior surface and an interior surface, wherein the cuff body defines a nerve channel for receiving a portion of a nerve; a lead body coupled, or coupleable, to the cuff body; at least one light emitter disposed on or within the cuff body or the lead body; and at least one reflective element disposed on, within, or beneath the interior surface of the cuff body, wherein the at least one reflective element is configured to reflect light emitted from the at least one light emitter. Alternatively or additionally, the cuff lead can include a receptacle for removably receiving a distal end portion of the lead body. Another system includes a cuff body with at least one light emitter, an electronic subassembly for operation, and an antenna to receive power from an external source.

### CROSS-REFERENCE TO RELATED APPLICATIONS

This application claims the benefit under 35 U.S.C. § 119(e) of U.S. Provisional Patent Application Ser. No. 63/433,874, filed Dec. 20, 2022, which is incorporated herein by reference.

### GOVERNMENT LICENSE RIGHTS

This invention was made with government support under 1R01NS121372 awarded by NIH—National Institute of Neurological Disorders and Stroke. The government has certain rights in the invention.

### FIELD

The present invention is directed to the area of implantable optical modulation systems and methods of making and using the systems. The present invention is also directed to implantable optical modulation cuff devices and systems, as well as methods of making and using the same.

### BACKGROUND

Implantable neuromodulation systems have proven therapeutic in a variety of diseases and disorders. Photobiomodulation (PBM) or other optical modulation can also provide therapeutic benefits in a variety of diseases and disorders by itself or in combination with electrical stimulation. A PBM system may include one or more light sources and, often, one or more optical fibers to carry the light to the desired modulation site. For example, spinal cord stimulation systems have been used as a therapeutic modality for the treatment of chronic pain syndromes. Peripheral nerve stimulation has been used to treat chronic pain syndrome and incontinence. Stimulation of the brain, such as deep brain stimulation, can be used to treat a variety of diseases or disorders.

### BRIEF SUMMARY

One aspect is an optical lead that includes a cuff body having an exterior surface and an interior surface, wherein the cuff body defines a nerve channel for receiving a portion of a nerve; a lead body coupled, or coupleable, to the cuff body; at least one light emitter disposed on or within the cuff body or the lead body; and at least one reflective element disposed on, within, or beneath the interior surface of the cuff body, wherein the at least one reflective element is configured to reflect light emitted from the at least one light emitter. It will be recognized that the at least one reflective element may also be used in any of the other aspects or embodiments described herein.

In at least some aspects of any of the embodiments described herein, the light emitter is a light source. In at least some aspects of any of the embodiments described herein, the light emitter is an emission region of an optical fiber, fiber optic, or other optical waveguide.

In at least some aspects, the at least one reflective element comprises a reflective foil, reflective coating, or reflective particles. In at least some aspects, the at least one reflective element includes a reflective sheet disposed on the interior surface of the cuff body. In at least some aspects, the at least one reflective element includes a plurality of reflective strips disposed on the interior surface of the cuff body. In at least some aspects, the at least one reflective element includes a reflective layer and the cuff body includes two polymeric layers with the reflective layer disposed therebetween. In at least some aspects, the at least one reflective element comprises a plurality of electrodes disposed on or with the cuff body, wherein the lead body includes a plurality of conductors coupled to the electrodes to provide electrical stimulation.

Another aspect is an optical lead that includes a cuff body having an exterior surface and an interior surface, wherein the cuff body defines a nerve channel for receiving a portion of a nerve; a lead body coupled, or coupleable, to the cuff body and including a distal end portion, wherein the cuff body includes a receptacle for removably receiving the distal end portion of the lead body, wherein the lead body and cuff body are capable of being coupled via the receptacle during a surgical procedure; and at least one light emitter disposed on or within the cuff body or the lead body. It will be recognized that the receptacle may also be used in any of the other aspects or embodiments described herein.

In at least some aspects, the receptacle is disposed on the interior surface of the cuff body. In at least some aspects, the receptacle is disposed on the exterior surface of the cuff body. In at least some aspects, the optical lead further includes a fastener configured to fasten the distal end portion of the lead body to the receptacle. In at least some aspects, at least of the at least one light emitter is disposed on or within the distal end portion of the lead body.

In at least some aspects, the lead body further includes a redirection element disposed within the distal end portion of the lead body and configured to receive light from the at least one of the at least one light emitter and redirect the received light. In at least some aspects, the cuff body further includes a redirection element disposed within the receptacle of the cuff body and configured to receive light from the at least one of the at least one light emitter, when the distal end portion of the lead body is received in the receptacle, and redirect the received light into the nerve channel.

In at least some aspects, the distal end portion of the lead body and the receptacle jointly form a tongue-and-groove arrangement to maintain the coupling of the lead body and the receptacle.

In at least some aspects of any of the embodiments described herein, the cuff body has a spiral arrangement for self-sizing of the cuff body around a nerve. In at least some aspects of any of the embodiments described herein, the optical lead further includes at least one suture tab, suture sleeve, or lead anchor configured for attaching the cuff body or lead body to tissue.

Another aspect is a system that includes any of the optical leads described above and a control module coupled, or coupleable, to the optical lead and configured to direct intermittent delivery of light via the at least one light emitter.

A further aspect is a system that includes a cuff body having an exterior surface and an interior surface, wherein the cuff body defines a nerve channel for receiving a portion of a nerve; at least one light emitter disposed on or within the cuff body; an electronic subassembly disposed on or within the cuff body and configured to direct intermittent delivery of light via the at least one light emitter; and an antenna disposed on or within the cuff body and coupled to the electronic subassembly and at least one light emitter for providing power from an external source.

### BRIEF DESCRIPTION OF THE DRAWINGS

Non-limiting and non-exhaustive embodiments of the present invention are described with reference to the following drawings. In the drawings, like reference numerals refer to like parts throughout the various figures unless otherwise specified.

For a better understanding of the present invention, reference will be made to the following Detailed Description, which is to be read in association with the accompanying drawings, wherein:

FIG. 1 is a schematic view of one embodiment of an optical or optical/electrical modulation system that includes a lead electrically coupled to a control module;

FIG. 2A is a schematic view of one embodiment of the control module of FIG. 1 configured and arranged to electrically couple to a lead body or other elongated device;

FIG. 2B is a schematic view of one embodiment of a lead extension configured and arranged to electrically couple a lead body or other elongated device to a control module;

FIG. 3A is a schematic perspective view of a distal end portion of one embodiment of an optical lead with a lead body and a cuff body;

FIG. 3B is a schematic top view of one embodiment of an interior surface of a cuff body with strips of a reflective element disposed thereon;

FIG. 3C is a schematic cross-sectional view of one embodiment of layers of a cuff body;

FIG. 4A is a schematic perspective view of a distal end portion of another embodiment of an optical lead with a lead body and a cuff body with a receptacle for receiving the lead body on the interior surface of the cuff body;

FIG. 4B is a schematic perspective view of the cuff lead of FIG. 4A with the lead body coupled to the cuff body;

FIG. 5 is a schematic perspective view of a distal end portion of a further embodiment of an optical/electrical lead with a lead body and a cuff body with electrodes disposed on the interior surface of the cuff body;

FIG. 6 is a schematic perspective view of a distal end portion of a yet another embodiment of an optical/electrical lead with a lead body and a cuff body with electrodes disposed on the interior surface of the cuff body at opposite ends;

FIG. 7 is a schematic perspective view of a distal end portion of a further embodiment of an optical/electrical lead with a lead body and a cuff body with a receptacle for the lead body on an exterior surface of the cuff body;

FIG. 8A is a schematic cross-sectional view of a portion of the lead body and cuff body of FIG. 7 with a mirror or other redirection element in the lead body;

FIG. 8B is a schematic cross-sectional view of a portion of the lead body and cuff body of FIG. 7 with a mirror or other redirection element in the receptacle of the cuff body;

FIG. 9 is a schematic perspective view of a distal end portion of another embodiment of an optical/electrical lead with a cuff body and a lead body coupled at an angle to the cuff body;

FIGS. 10A and 10B are schematic perspective views of a distal end portion of a yet another embodiment of an optical lead with a lead body and a cuff body in a spiral arrangement to facilitate fitting over nerves or tissue of different diameters;

FIG. 11 is a schematic perspective view of a distal end portion of a further embodiment of an optical lead with a lead body and a cuff body and fixation elements for maintaining the position of the lead body or cuff body relative to tissue;

FIG. 12 is a schematic partial, top view of a distal end portion of another embodiment of an optical lead illustrating a tongue-and-groove arrangement for coupling the lead body to a receptacle of the cuff body;

FIG. 13 is a schematic perspective view of a distal end portion of one embodiment of a system with a cuff body, one or more light emitters, an electronic subassembly, and an antenna; and

FIG. 14 is a schematic overview of one embodiment of components of an optical or optical/electrical modulation arrangement.

### DETAILED DESCRIPTION

The present invention is directed to the area of implantable optical modulation systems and methods of making and using the systems. The present invention is also directed to implantable optical modulation cuff devices and systems, as well as methods of making and using the same.

In some embodiments, an implantable optical modulation system only provides optical modulation, such as optical stimulation or optical inhibition. Examples of optical modulation systems with leads are found in, for example, U.S. Pat. Nos. 9,415,154; 10,335,607; 10,625,072; and 10,814,140 and U.S. Patent Applications Publication Nos. 2020/0155584; 2020/0376272; 2021/0008388; 2021/0008389; 2021/0016111; and 2022/0072329, all of which are incorporated by reference in their entireties. Any of these leads can be adapted to provide a cuff lead.

In other embodiments, the stimulation system can provide both optical modulation and electrical stimulation. In at least some of these embodiments, the optical modulation system can be a modification of an electrical stimulation system to also provide optical modulation. Suitable implantable electrical stimulation systems that can be modified to also provide optical modulation include, but are not limited to, a least one lead with one or more electrodes disposed along a distal end of the lead and one or more terminals disposed along the one or more proximal ends of the lead. Any of these leads can be adapted to provide a cuff lead. Examples of electrical stimulation systems with leads are found in, for example, U.S. Pat. Nos. 6,181,969; 6,516,227; 6,609,029; 6,609,032; 6,741,892; 7,203,548; 7,244,150; 7,450,997; 7,596,414; 7,610,103; 7,672,734;7,761,165; 7,783,359; 7,792,590; 7,809,446; 7,949,395; 7,974,706; 6,175,710; 6,224,450; 6,271,094; 6,295,944; 6,364,278; and 6,391,985; U.S. Patent Applications Publication Nos. 2007/0150036; 2009/0187222; 2009/0276021; 2010/0076535; 2010/0268298; 2011/0004267; 2011/0078900; 2011/0130817; 2011/0130818; 2011/0238129; 2011/0313500; 2012/0016378; 2012/0046710; 2012/0071949; 2012/0165911; 2012/0197375; 2012/0203316; 2012/0203320; 2012/0203321; 2012/0316615; and 2013/0105071; and U.S. patent applications Ser. Nos. 12/177,823 and 13/750,725, all of which are incorporated by reference in their entireties.

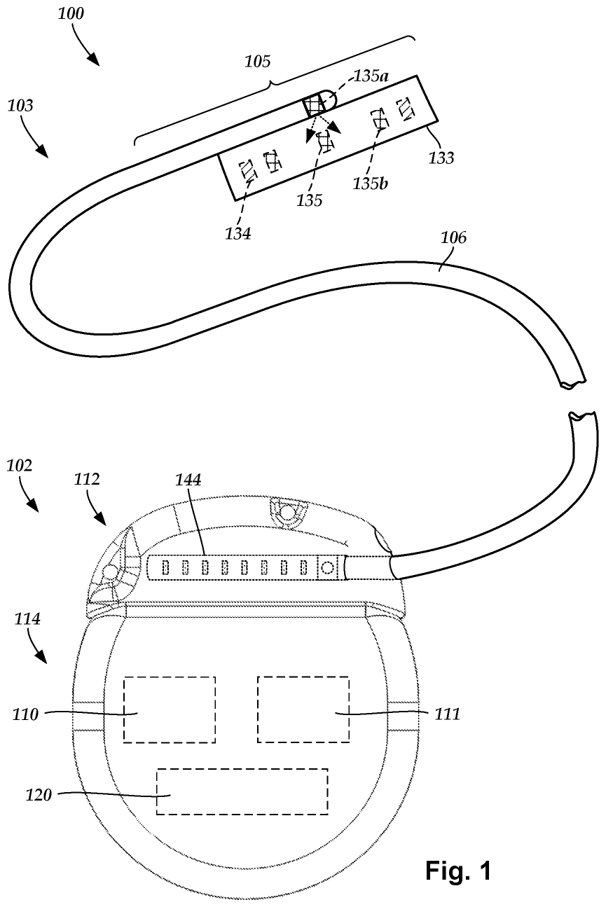

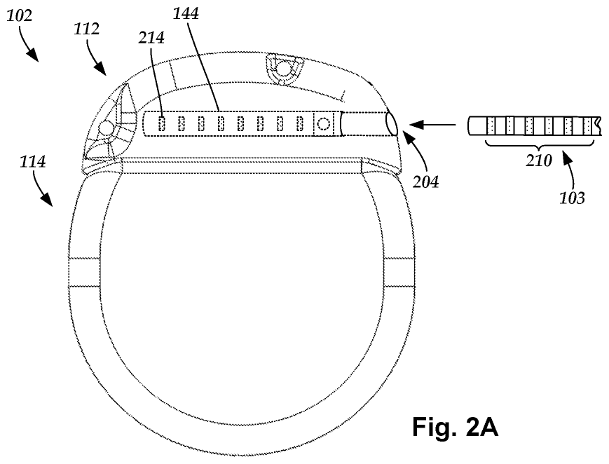

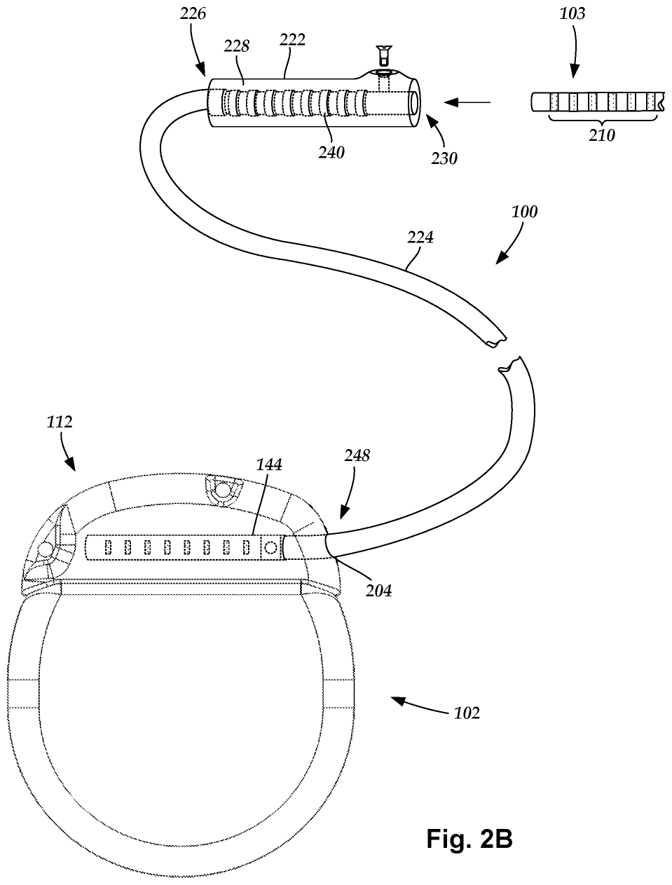

FIG. 1 illustrates schematically one embodiment of an optical modulation system 100. The optical modulation system includes a control module (e.g., a stimulator or pulse generator) 102 and a lead 103 coupleable to the control module 102. The lead 103 includes one or more lead bodies 106, at least one cuff body 133 coupled to a distal end portion 105 of a lead body 106, one or more light emitters 135, one or more optional electrodes 134, and one or more terminals or light receivers (e.g., 210 in FIG. 2A-2B) disposed along the one or more lead bodies 106. In at least some embodiments, the lead is isodiametric along a longitudinal length of the lead body 106.

In at least some embodiments, one or more (or all) of the light emitters 135 can be a light source, such as a light emitting diode (LED), organic light emitting diode (OLED), laser diode, or the like or any combination thereof. In at least some embodiments, conductors 129 (FIGS. 8A and 8B) extending along the lead 103 and from the control module 102 to provide signals and power for operating the light source. When there are multiple light sources, the light emitted by the light sources can have a same wavelength or wavelength band or some, or all, of the light sources can emit light at different wavelengths or different wavelength bands.

In at least some embodiments, one or more of the light emitters 135 can be a terminus or other light emitting region of an optical fiber, fiber optic, optical waveguide, or the like. In such embodiments, one or more light sources can be disposed in the control module 102, the lead body 106, or in any other suitable structure (such as an adapter, lead extension, lead extension connector, or the like) that can provide light to the optical fiber, fiber optic, optical waveguide, or the like for emission at the light emitters 135. Examples of light sources disposed in these components can be found in the references cited above.

In at least some embodiments, the light emitter(s) 135 can be disposed on or in the cuff body 133, as illustrated by light emitter 135b. In at least some embodiments, the light emitter(s) 135 can be disposed on or in a portion of the lead body 106 that contacts the cuff body 133, as illustrated by light emitter 135a, and illuminate a nerve or other tissue disposed within the cuff body. Any combination of light emitters 135a, 135b can be used.

The lead 103 can be coupled to the control module 102 in any suitable manner. In some embodiments, the lead is permanently attached to the control module 102. In other embodiments, the lead can be coupled to the control module 102 by a connector 144. In at least some embodiments, the lead 103 couples directly to the control module 102. In at least some other embodiments, the lead 103 couples to the control module 102 via one or more intermediate devices (such as the lead extension 224 of FIG. 2B). For example, in at least some embodiments one or more lead extensions 224 (FIG. 2B) can be disposed between the lead 103 and the control module 102 to extend the distance between the lead 103 and the control module 102. Other intermediate devices may be used in addition to, or in lieu of, one or more lead extensions including, for example, a splitter, an adaptor, or the like or combinations thereof. It will be understood that, in the case where the optical modulation system 100 includes multiple intermediate devices disposed between the lead 103 and the control module 102, the intermediate devices may be configured into any suitable arrangement.

The control module 102 typically includes a connector housing 112 and a sealed electronics housing 114. Stimulation circuitry 110 and an optional power source 120 are disposed in the electronics housing 114. A control module connector 144 is disposed in the connector housing 112. The control module connector 144 is configured and arranged to make an electrical connection between the lead 103 and the stimulation circuitry 110 of the control module 102.

In some embodiments, the control module 102 also includes one or more light sources 111 disposed within the sealed electronics housing 114. The one or more light sources can be, for example, a light emitting diode (LED), laser diode, organic light emitting diode (OLED), or the like. When the control module 102 includes multiple light sources, the light sources can provide light at a same wavelength or wavelength band or some, or all, of the light sources can provide light at different wavelengths or different wavelength bands. When the control module includes one or more light sources 111, the light emitted by the light sources can be directed to an optical fiber, a series of optical fibers, or other light transmitting body(ies). The optical fiber, series of optical fibers, or other light transmitting body(ies) can transmit the light from the one or more light sources 111 through the control module 102 and lead 103 to the light emitter(s) 135 (which can be a terminus or other light emitting region of the optical fiber). In at least some embodiments, the optical fiber is a single mode optical fiber. In other embodiments, the optical fiber is a multi-mode optical fiber. In some embodiments, the system includes a single optical fiber. In other embodiments, the system may employ multiple optical fibers in series or in parallel.

The optical modulation system or components of the optical modulation system, including the lead body 106, cuff body 133, and the control module 102, are typically implanted into the body of a patient. In at least some embodiments, the cuff body 133 is implanted with a portion of a nerve or other neural tissue disposed within the cuff body and, typically, extending out one or both ends of the cuff body.

If the lead includes the optional electrodes 134, the electrodes can be formed using any conductive, biocompatible material. Examples of suitable materials include metals, alloys, conductive polymers, conductive carbon, and the like, as well as combinations thereof. In at least some embodiments, one or more of the electrodes 134 are formed from one or more of: platinum, platinum iridium, palladium, palladium rhodium, iridium, iridium oxide, or titanium. Any suitable number of electrodes 134 can be disposed on the lead including, for example, one, two, three, four, five, six, seven, eight, nine, ten, eleven, twelve, fourteen, sixteen, twenty-four, thirty-two, or more electrodes 134.

The one or more lead bodies 106 are made of a non-conductive, biocompatible material such as, for example, silicone, polyurethane, polyether ether ketone (“PEEK”), epoxy, and the like or combinations thereof. The one or more lead bodies 106 may be formed in the desired shape by any process including, for example, molding (including injection molding), casting, and the like.

One or more terminals 210 (FIGS. 2A-2B) are typically disposed along the proximal end of the one or more lead bodies 106 of the stimulation system 100 (as well as any splitters, lead extensions, adaptors, or the like) for electrical connection to corresponding connector contacts 214 (FIG. 2A). The connector contacts are disposed in connectors 144 which, in turn, are disposed on, for example, the control module 102 (or a lead extension, a splitter, an adaptor, or the like). Electrically conductive wires, cables, or the like (not shown) extend from the terminals to the light emitter 135 or optional one or more electrodes 134. One or more of the terminals can be replaced by light receivers (for example, terminal ends of optical fibers, fiber optics, or light waveguides) to receive light from the light source(s) 111 in the control module 102, when present.

The electrically conductive wires, optical fibers, fiber optics, or optical waveguides (“conductors” 129—FIGS. 8A and 8B) may be embedded in the non-conductive material of the lead body 106 or can be disposed in one or more lumens (not shown) extending along the lead body 106. In some embodiments, there is an individual lumen for each conductor. In other embodiments, two or more conductors 129 (FIGS. 8A and 8B) extend through a lumen. There may also be one or more lumens (not shown) that open at, or near, the proximal end of the lead body 106, for example, for inserting a stylet to facilitate placement of the lead body 106 within a body of a patient. Additionally, there may be one or more lumens (not shown) that open at, or near, the distal end of the lead body 106, for example, for infusion of drugs or medication into the site of implantation of the lead body 106. In at least one embodiment, the one or more lumens are flushed continually, or on a regular basis, with saline, epidural fluid, or the like. In at least some embodiments, the one or more lumens are permanently or removably sealable at the distal end.

In FIGS. 2A and 2B, the connector housing 112 is shown having one port 204. The connector housing 112 can define any suitable number of ports including, for example, one, two, three, four, five, six, seven, eight, or more ports. The control module connector 144 also includes a plurality of connector contacts, such as connector contact 214, disposed within each port 204. When the lead 100 is inserted into the port 204, the connector contacts 214 can be aligned with a plurality of terminals 210 disposed along the proximal end(s) of the lead 100 to electrically couple the control module 102 to the electrodes (134 of FIG. 1) and optionally the light emitters 135 (particularly if the light emitter(s) is/are light sources) disposed at a distal end of the lead 103. Examples of connectors in control modules are found in, for example, U.S. Pat. Nos. 7,244,150 and 8,224,450, which are incorporated by reference.

FIG. 2B is a schematic side view of another embodiment of the optical stimulation system 100. The optical stimulation system 100 includes a lead extension 224 that is configured and arranged to couple one or more elongated devices (for example, the lead body 106, an adaptor, another lead extension, or the like or combinations thereof) to the control module 102. In FIG. 2B, the lead extension 224 is shown coupled to a single port 204 defined in the control module connector 144. Additionally, the lead extension 224 is shown configured and arranged to couple to a lead 100 or other elongated device. In alternate embodiments, the lead extension 224 is configured and arranged to couple to multiple ports 204 defined in the control module connector 144, or to receive multiple leads 100, or both.

A lead extension connector 222 is disposed on the lead extension 224. In FIG. 2B, the lead extension connector 222 is shown disposed at a distal end 226 of the lead extension 224. The lead extension connector 222 includes a connector housing 228. The connector housing 228 defines at least one port 230 into which terminals 210 of the lead 100 or other elongated device can be inserted. The connector housing 228 also includes a plurality of connector contacts, such as connector contact 240. When the lead 100 or other elongated device is inserted into the port 230, the connector contacts 240 disposed in the connector housing 228 can be aligned with the terminals 210 of the lead 100 or other elongated device to electrically couple the lead extension 224 to the electrodes (134 of FIG. 1) and optionally the light emitter(s) 135 disposed along the lead (103 in FIG. 1).

In at least some embodiments, the proximal end of the lead extension 224 is similarly configured and arranged as a proximal end of the lead 103 (or other elongated device). The lead extension 224 may include a plurality of electrically conductive wires (not shown) that electrically couple the connector contacts 240 to a proximal end 248 of the lead extension 224 that is opposite to the distal end 226. In at least some embodiments, the conductive wires disposed in the lead extension 224 can be electrically coupled to a plurality of terminals (not shown) disposed along the proximal end 248 of the lead extension 224. In at least some embodiments, the proximal end 248 of the lead extension 224 is configured and arranged for insertion into a connector disposed in another lead extension (or another intermediate device). In other embodiments (and as shown in FIG. 2B), the proximal end 248 of the lead extension 224 is configured and arranged for insertion into the control module connector 144.

Systems and methods are described herein that include cuff designs for optical modulation or photobiomodulation (PBM) (including targeted PBM) of nerves or other tissue. The systems and methods can have one or more of the following properties as compared to conventional optical modulation or PBM systems: energy efficiency (for example, by increasing the probability that photons are absorbed in the target tissue such as a target nerve); reduce or minimize heating (for example, by using a wavelength that causes a desired effect that persists for a duration greater than the duration of light application); controlled delivery (for example, by controlling the spatial arrangement of light delivery); avoid or reduce unwanted effects to surrounding tissues (for example, by limiting the amount of light and heat that is absorbed by non-target tissues); application to nerves of different sizes (for example, by using a construction that can fit different sizes of nerves); fixation to manage mechanical insult to the nerve (for example, by including fixation features to reduce mechanical strain on a nerve or nerve bundle); avoid being too tight on the nerve (for example, by using self-sizing); safe or easy replacement of optical delivery device without disrupting nerve interface (for example, using a module design to facilitate placement and replacement); support continuous and non-continuous energy delivery models (for example, by using PBM parameters that have an effect that can persist following bolus delivery); support various user-friendly use models (for example using an implantable energy source or transcutaneous delivery of operating energy); improved reduction in pain with a single nerve cuff device (for example, using two modes of treatment—optical modulation/PBM and electrical stimulation); or any combination thereof.

As an example, in at least some instances, a cuff lead delivering PBM can be used to selectively disrupt small fiber (e.g., afferent) activity while leaving large fiber activity intact in mixed nerves. As another example, chronic application of PBM to a peripheral nerve can provide therapy for treatment of pain syndromes because the small fibers that transmit pain information can be “turned down” or “turned off” (i.e., PBM used to reduce or halt the transmission of pain information) without disrupting other functions such as, for example, proprioception, mechanosensory function, or motor control.

Other therapies that involve disrupting action potential propagation in small fibers (selective or non-selective) are also available. For example, PBM can be used to suppress activity of a neural circuit that is pathologically active. As another example, PBM can be used to disinhibit activity of a pathologically depressed neural circuit by suppressing activity of an inhibitory input.

Optical modulation or PBM can also be used to achieve other effects, such as, for example, anti-inflammatory effects, healing promotion, neuroprotective effects, or the like. The systems and methods described herein may be useful in achieving these effects. In at least some embodiments, such effects (for example, inhibitory effects) can endure beyond the application of light.

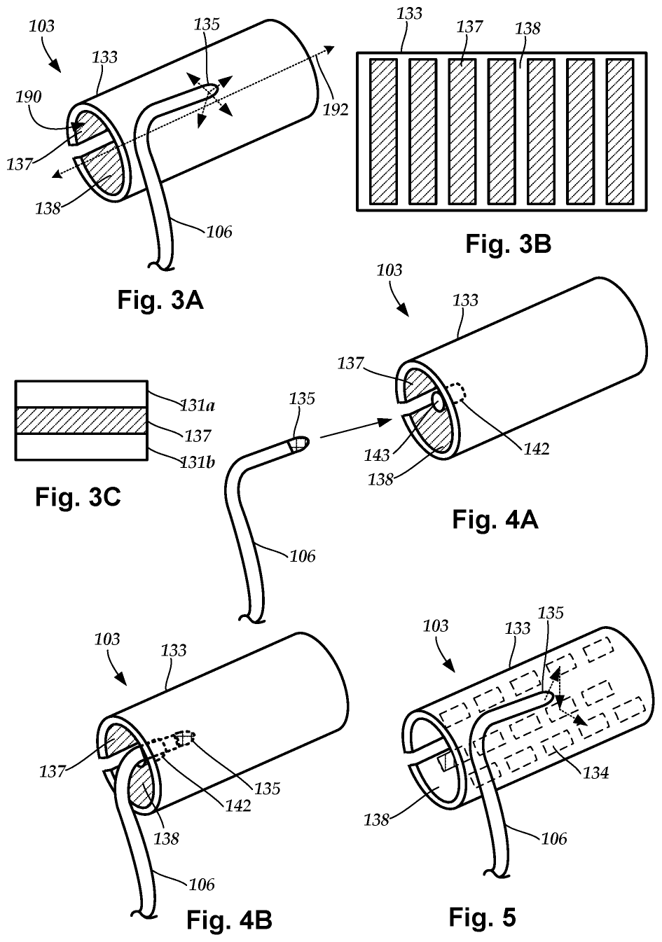

In at least some embodiments, the systems and methods described herein utilize a cuff lead with a cuff body that receives a portion of a nerve or other tissue within the cuff body. The use of a cuff can reduce or limit stimulation or PBM of tissue outside of the cuff body. FIG. 3A illustrates one embodiment of a distal portion of a cuff lead 103 that includes a cuff body 133, a lead body 106 coupled to the cuff body, and one or more light emitters 135 disposed on or in the cuff body or lead body. In the embodiment illustrated in FIG. 3A, a light emitter 135 is disposed in the lead body 106 and emits light within the cuff body 133. The cuff body 133 defines a nerve channel 190 having a nerve channel axis 192.

In at least some embodiments, the cuff body 133 includes a reflective element 137 disposed on or within an interior surface 138 of the cuff body. The reflective element 137 can be a reflective foil, a reflective coating, reflective particles on or in the cuff body, or any other suitable reflective arrangement or any combination thereof. Any suitable, biocompatible light reflective material can be used and may be selected based on the wavelength(s) of light that are emitted from the light emitters 135. Examples of light reflective materials include, but are not limited to, foils or coatings of biocompatible metals, such as gold, platinum, or titanium, or biocompatible metal alloys, such as platinum iridium, as well as foils or coatings of biocompatible nanomaterials, such as graphene, borophene, or biocompatible polymers, such as retroreflective foils made using oriented birefringent polymers. In at least some embodiments, the reflective element 137 reflects at least 25, 50, 60, 75, 80, or 90% of light of a particular wavelength or range of wavelengths that illuminates the reflective element. In at least some embodiments, the reflective element 137 is not coupled, or coupleable, to a power source in normal operation of the cuff lead 103 or is not an electrode. The reflective element 137 can be used with any of the other embodiments of cuff leads 103 described herein.

The reflective element 137 can be a single piece, as illustrated in FIG. 3A, or multiple pieces distributed over the interior surface of the cuff body 133. FIG. 3B illustrates one embodiments of an unrolled cuff body 133 and a reflective element 137 formed of multiple strips 139 of reflective material attached to an interior surface 138 of the cuff body. In at least some embodiments, the use of strips 139 (or other multi-piece arrangement) instead of a single sheet may increase flexibility of the combination of cuff body 133 and reflective element.

The reflective element 137 can be disposed on the cuff body 133, as illustrated in FIGS. 3A and 3B. In at least some embodiments, the reflective element 137 can be disposed between polymer layers 131a, 131b of the cuff body 133, as illustrated in cross-section in FIG. 3C.

In at least some embodiments, the reflective element 137 can reflect light that would otherwise be transmitted through, absorbed by, or scattered by the cuff body 133. In at least some embodiments, the reflective element 137 can increase a percentage of the light from the light emitter(s) 135 that interacts with, or is absorbed by, the nerve or other tissue within the cuff body 133 as compared to a cuff body without the reflective element because light is reflected back toward the nerve or other tissue. In at least some embodiments, use of the reflective element 137 can reduce the amount of light needed to elicit a desired effect as compared to a cuff body without the reflective element due to reflection of light back toward the nerve or other tissue. In at least some embodiments, use of the reflective element 137 can reduce the amount of light leakage (or heat leakage or both) to surrounding tissues as compared to a cuff body without the reflective element. Such a reduction can reduce or limit PBM of surrounding tissue or the heating of the surrounding tissues.

In at least some embodiments, use of the reflective element 137 can increase the uniformity of light distribution through the portion of the nerve within the cuff body 133 as compared to a cuff body without the reflective element. In at least some embodiments, use of the reflective element 137 can reduce the sensitivity to rotation of the cuff body 133 as compared to a cuff body without the reflective element.

FIGS. 4A and 4B illustrate another embodiment of a distal portion of a cuff lead 103 that includes a cuff body 133, a lead body 106, and at least one light emitter 135 disposed on the distal portion of the lead body. In at least some embodiments, the lead body 106 can be a percutaneous lead with at least one light emitter 135 and the cuff body 133 can act as an anchor or holder for the percutaneous lead.

In at least some embodiments, the cuff body 133 includes a receptacle 142 disposed on an interior surface of the cuff body. The receptacle 142 receives a portion of the lead body 106 into an opening 143 of the receptacle. In at least some embodiments, there may be two or more receptacles 142 or the receptacle may extend along at least 5 or 10% of the length of the cuff body 133 to stabilize the lead body 106 when received. In at least some embodiments, a fastener, such as a set screw 146 (FIG. 7), can engage the lead body 106 and the cuff body 133 for retention of the lead body. Any other suitable fastener (for example, adhesive or the like) or fastening mechanism (for example, friction fit, a septum, or the like) can be used. Alternatively or additionally, the lead body 106 and cuff body 133 can be attached to the tissue using sutures, suture tabs, suture sleeves, or the like or any combination thereof as described below. Any of the other cuff leads 103 disclosed herein can include a receptacle 142. In at least some embodiments, the receptacle 142 can be in the cuff body 133, such as an opening in the cuff body.

In at least some embodiments, the cuff body 133 includes a reflective element 137 disposed on or in the cuff body, as described above. The reflective element 137 can facilitate distribution of the light from the light emitter(s) 135 of the lead body 106.

FIG. 5 illustrates a distal portion of a cuff lead 103 that includes a cuff body 133, one or more light emitters 135 disposed on or in the cuff body or lead body, one or more electrodes 134 disposed on or in the cuff body, and a lead body 106 coupled to the cuff body. The electrodes 134 can be used for electrical stimulation, electrical recording, or both and can be coupled to a control module 102 via conductors in the lead body 106 (or another lead body).

In at least some embodiments, the material and finish of the electrodes 134 are selected so that the reflectance of the electrodes 134 for one or more selected wavelengths of light emitted by the light emitters is at least 50, 60, 70, 75, or 80 percent or more. For example, for at least some surface finishes, platinum reflectance can be at least 70% for 810 nm light with incidence angles greater than 30 degrees. In at least some embodiments, an array of electrodes 134 can be placed around the interior surface of the cuff body 133. The electrodes 134 can act as the reflective element 137, as described above. Alternatively or additionally, a separate reflective element 137, such as a metal foil, can be positioned on the cuff body 133 to increase reflectance of light.

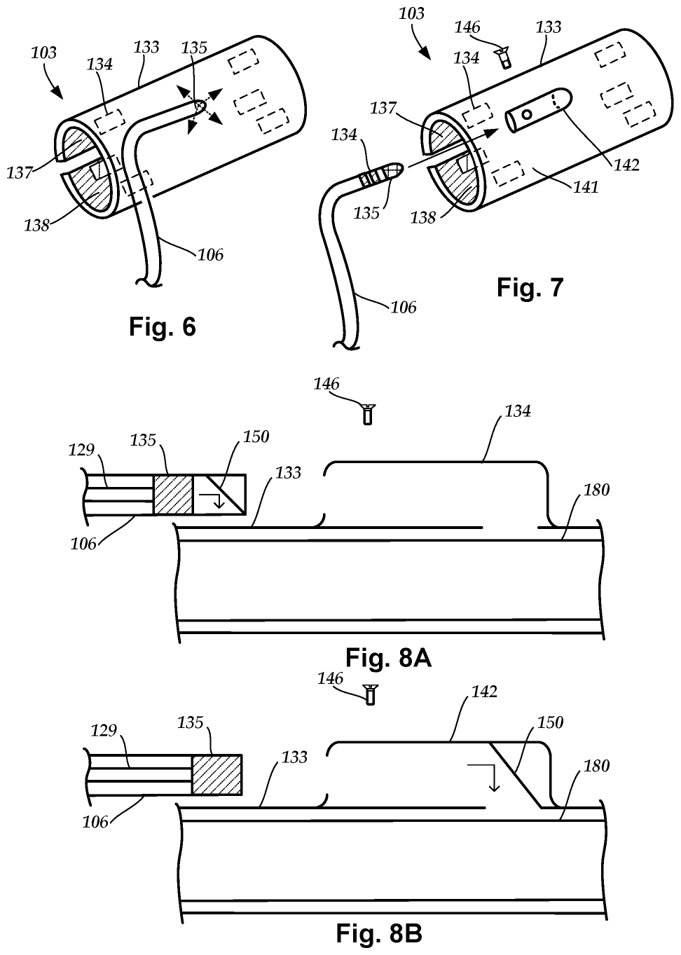

FIG. 6 illustrates a distal portion of another cuff lead 103 that includes a cuff body 133, light emitters 135 disposed on or in the cuff body, electrodes 134 disposed on or in the cuff body, and a lead body 106 coupled to the cuff body. In this embodiment, a PBM delivery region is shown in the center of the cuff and electrodes 134 are shown on both ends (or optionally only one of the ends) of the cuff body 133. Optionally, a reflective element 137, such as a reflective foil, can be provided at the PBM delivery region, but may or may not be provided over or near the electrodes 134. In at least some embodiments, a lead body 106 can include one or more light emitters 135 and one or more electrodes 134, as illustrated in FIG. 7.

Returning to FIG. 6, in at least some embodiments, the light emitter(s) 135 are coupled to the control module 102 using a separate lead body 106 from a lead body used to provide electrical stimulation via the electrodes 134. In other embodiments, the same lead body 106 can be used for coupling the electrodes 134 and the light emitter(s) 135 to the control module 102.

In at least some embodiments, optical modulation/PBM and electrical stimulation can be used simultaneously or sequentially. For example, these two different modes can provide two different methods to reduce nociceptive information that is conveyed to the brain, and exploit both from a single implantable stimulation system. As an example, c-fiber input can be reduced in a peripheral nerve by using PBM to block or reduce the ability of c-fibers to convey action potentials to the spinal cord. Electrical stimulation of the larger AB fibers can act to “close” the gate (for example, a neural circuit in the dorsal horn of the spinal cord) on transmission of smaller fiber activity through the circuit and on to the brain.

In at least some embodiments, the electrodes 134 at one or both ends of the cuff body 133 can be used for sensing of the compound action potentials (CAP). CAP from different locations (for example, at upstream and downstream locations flanking the PBM site) can be analyzed (for example, decomposed for activities of different fiber size) and compared to evaluate the effect of PBM.

In at least some embodiments, when a cuff body 133 is of adequate length, stimulation can be provided at one end of the cuff body and an evoked compound action potential can be recorded by one or more electrodes 134 at the other end of the cuff body. Analyzing this response can facilitate determining a degree to which small fiber activity (or even large fiber activity) has been blocked by the PBM effect. Alternatively or additionally, in at least some embodiments, an additional cuff lead 103 or other electrode device (e.g., a spinal cord stimulation lead) can be used for the recording or stimulation.

In at least some embodiments, a signal can be measured at one location using one or more electrode 134. Based on the measured signal, illumination at the nerve can be initiated, halted, increased, or decreased.

Returning to FIG. 7, the cuff body 133 and the lead body 106 (with at least one light emitter 135) are separate components. In at least some embodiments, the lead body 106 is received in a receptacle 142 disposed on an exterior surface 141 of the cuff body 133. In at least some embodiments, the lead body 106 can be retained in the receptacle 142 using a set screw 146 or any other suitable fastener (for example, adhesive or the like) or fastening mechanism (for example, friction fit, a septum, or the like). In at least some embodiments, the use of a receptacle 142 (either on the exterior surface 141 or the interior surface 138, as illustrated in FIGS. 4A and 4B) can facilitate surgical placement of the cuff body 133 with later placement of the lead body 106. In at least some embodiments, the use of a receptacle 142 can facilitate replacement of the lead body 106 without removing the cuff body 133 from the nerve or other tissue. This may be particularly useful when replacement of the light emitter(s) 135 is needed.

FIGS. 8A and 8B illustrate two embodiments of a cuff body 133 disposed around a nerve 180 or other tissue, a receptacle 142 (which is illustrated on the exterior surface 141, as in FIG. 7, but can also be on the interior surface, as in FIGS. 4A and 4B), a lead body 106, a light emitter 135 disposed on or within the lead body, and conductors 129 extending along the lead body to provide power to the light emitter from the power source 120 (FIG. 1) in the control module 102 (FIG. 1). In at least some embodiments, a set screw 146 (or other fastener or fastening mechanism) is provided to fix the lead body 106 within the receptacle 142.

In at least some embodiments, as illustrated in FIG. 8A, the lead body 106 can include a redirection element 150 that receives light from the light emitter 135 and redirects the light toward a side of the lead body and, when the lead body is disposed in the receptacle 142 of the cuff body 133, illuminates the nerve 180 or other tissue within the cuff body. The redirection element 150 can be a mirror, prism, angled lens, or any other suitable reflector or element that redirects light into a different path. In at least some embodiments, the shape of the receptacle 142 and the distal end of the lead body 106 is selected to correctly orient the lead body within the receptacle so that the light is redirected into the cuff body 133.

Alternatively, in at least some embodiments, as illustrated in FIG. 8B, the receptacle 142 of the cuff body 133 can include a redirection element 150 or other light reflector that receives light from the light emitter 135 when the lead body 106 is inserted into the receptacle. The redirection element 150 or other light reflector redirects the light from the light emitter 135 on or within the lead body 106 to illuminate the nerve 180 or other tissue within the cuff body 133. In at least some embodiments, there is no need for shape restrictions on the lead body 106 or receptacle 142 to achieve alignment.

It will be understood that inclusion of a receptacle 142 on the interior surface 138 or exterior surface 141 of the cuff body 133 can be applied to any of the other cuff leads 103 described herein. It will be understood that a redirection element or other light reflector can be included in any of the lead bodies described herein or in any of the receptacles described herein.

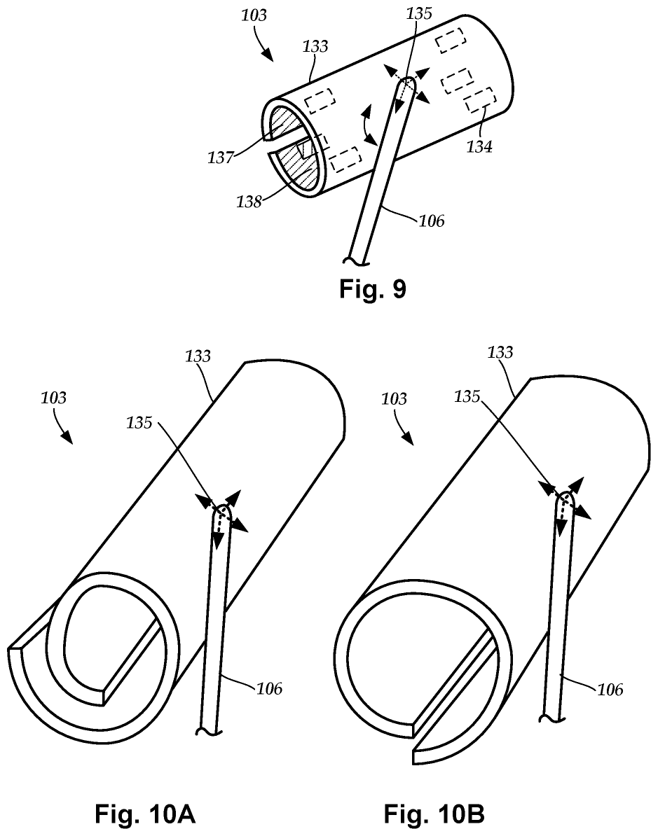

In at least some embodiments, as illustrated in FIG. 9, the lead body 106 includes a light emitter 135 and is attached (either permanently or removably) to the cuff body 133 at an angle 152 of at least 20, 25, 30, 45, 50, or 60 degrees or more with respect to the cuff body. Surgically, it may be easier and more mechanically stable to position the lead body 106 closer to parallel to the nerve 180 (FIGS. 8A and 8B) rather than perpendicular (although, in some instances, it may be preferable for surgical or anatomical reasons to angle the lead body with respect to the cuff body). However, if the lead body 106 is parallel, less of the light from the light emitter 135 may be directed toward the nerve. In at least some embodiments, presenting the lead body 106 at the angle 152 facilitates directing the light from the light emitter 135 toward the nerve 180 or other tissue disposed within the cuff body 133.

Nerves 180 (FIGS. 8A and 8B) can have different sizes (for example, diameters) and a cuff body 133 can be selected based on the diameter of the nerve or other tissue to be disposed within the cuff body. In at least some embodiments, the cuff body 133 can be designed for a variety of diameters, as illustrated in FIGS. 10A and 10B. In at least some embodiments, the cuff body 133 can be self-sizing. As illustrated in FIGS. 10A and 10B, the cuff body 133 can have a spiral arrangement. In at least some embodiments, the cuff body 133 can be made of a material that maintains the spiral arrangement and can expand in response to receiving the nerve or other tissue instead of cause compression damage. In at least some embodiments, the material and other features (for example, the thickness, spring constant, or the like) of the cuff body 133 can be made to balance retention of the spiral arrangement versus compression of the nerve or other tissue within the cuff body.

A cuff body 133 with a spiral arrangement, as illustrated in FIGS. 10A and 10B, can also be combined with a reflective element 137, as described above, or a receptacle 142, as described above, or any combination thereof. In at least some embodiments, addition of a reflective element 137, as described above, such as a reflective foil or reflective layer, can alter mechanical properties of a spiral arrangement. For example, a reflective foil or reflective layer can allow a clinician or other individual to shape the cuff body 103 which may better fit a nerve or other tissue and avoid or reduce compression.

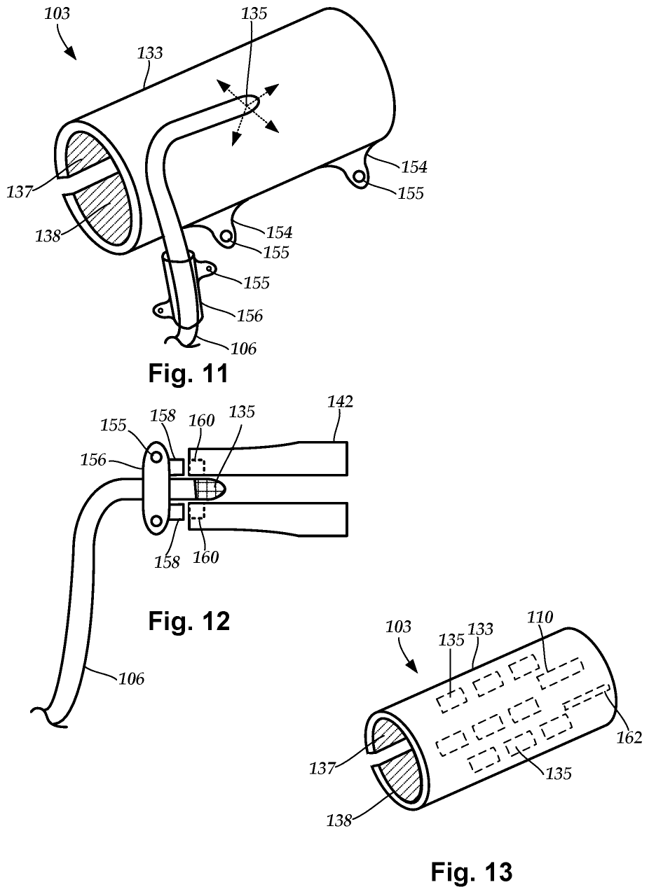

FIG. 11 illustrates a distal portion of a cuff lead 103 with one or more suture tabs 154 on the cuff body 133 to hold the cuff body in place with respect to the nerve and other surrounding tissue. Additionally or alternatively, a lead anchor or suture sleeve 156 can be attached to the lead body 106. Examples of lead anchors and suture sleeves can be found in U.S. Pat. Nos. 8,412,349; 8,467,883; 9,095,701; 9,636,498; 9,887,470; 9,987,482; 10,071,242; and 10,857,351 and U.S. Patent Application Publications Nos. 2014/0081366; 2018/0272125; and 2019/0105503, all of which are incorporated herein by reference in their entireties.

The suture tabs 154 and lead anchor or suture sleeve 156 can include suture openings 155 for threading a suture to affix the cuff body 133 or lead body 106 to tissue. The lead anchor or suture sleeve 156 may be removable from the lead body or permanently attached or coupled to the lead body. A removable lead anchor or suture sleeve 156 can be fixed to the lead body 106 with a suture, a set screw, adhesive, or the like or any of the mechanism described in the cited references. Any other suitable fixation elements can be used. In at least some embodiments, at least two fixation elements are arranged along, or parallel to, the nerve axis to facilitate maintaining the cuff body 133 aligned with the nerve 180. Such an arrangement may reduce or minimize torque on the nerve.

In at least some embodiments, a lead body 106 can include a suture sleeve 156 with one or more tongue elements 158 that fit into corresponding groove(s) 160 on the receptacle 142, as illustrated in FIG. 12. The receptacle 142 can be, for example, any of the receptacles illustrated in FIGS. 4A, 4B, 7, 8A, or 8B. It will be recognized that in other embodiments, the tongue element(s) 158 can be on the receptacle 142 and the groove(s) 160 can be in the suture sleeve 156. It will also be recognized that the suture sleeve 156 can include a combination of tongue element(s) 158 and groove(s) 160 with corresponding groove(s) and tongue element(s) on the receptacle 142. The tongue element(s) 158 fit in the groove(s) 160 to stabilize the lead body 106 and cuff body 133 and may be used for alignment of the lead body with the cuff body. In at least some embodiments, the receptacle 142 is tapered.

Another option for alignment of the lead body 106 within the cuff body 133 include marker alignment using marks on the lead body and the cuff body. Any other suitable alignment method or arrangement can be used. The alignment of the lead body 106 with the cuff body 133 may be particularly useful for those embodiments that utilize a redirection element 150 (see, FIGS. 8A and 8B) where proper alignment may increase transmission of light to the nerve or other tissue.

In at least some embodiments, the positioning of a suture sleeve 156 on the lead body 106 can be adjusted to a depth that the light emitter 135 or lead body 106 extends into, or through, the receptable 142 or cuff body 133. In at least some embodiments, the suture sleeve 156 can provide a stop function to prevent or hinder the lead body 106 from going too far into the cuff body.

In at least some embodiments, a relatively short duration of PBM application (for example, seconds, minutes) can result in relatively long duration effects (for example, hours, days, or weeks). In at least some embodiments, the therapy can be delivered by periodic powering of the device for PBM delivery. In at least some embodiments, as illustrated in FIG. 13, an antenna 162 can be included in the cuff body 133 (or, alternatively, a lead body 106 (FIG. 3A)) and coupled to the light emitter(s) 135 for powering the light emitter(s). In some embodiments, the cuff body 133 (or, alternatively, a lead body 106 (FIG. 3A)) may also contain an electronic subassembly (such as, for example, electronic subassembly 110 of FIG. 1) to regulate light delivery. In such embodiments, a control module 102 may be unnecessary as the electronic subassembly (FIG. 1) resides in the cuff and power is supplied via the antenna 162. In at least some embodiments, external signals (for example, from an RF source) may provide both power and information.

In at least some embodiments, an optical or optical/electrical modulation system may only include a cuff body 133 having the light emitter(s) 135, antenna 162, electronics subassembly 110, and optional electrode(s) 134. Such an arrangement may result in less stress on the nerve as there is no connection to a lead body 106. Other advantages can include the lack of an implantable power source (which may reduce cost or size), the lack of lead connections between components, no need to replace the power source when it is depleted, or the like. In at least some embodiments, a user positions an external power source near to the cuff periodically or when need (for example, a few times daily, weekly, or monthly, or as needed) to provide therapy by inductively coupling the external power source to the antenna 162.

In at least some embodiments, a substance is placed in or on the cuff body 133 to absorb energy to provide heat to the nerve 180. For example, gold nanoparticles can be embedded in the cuff and light can be used to illuminate the gold nanoparticles to generate heat.

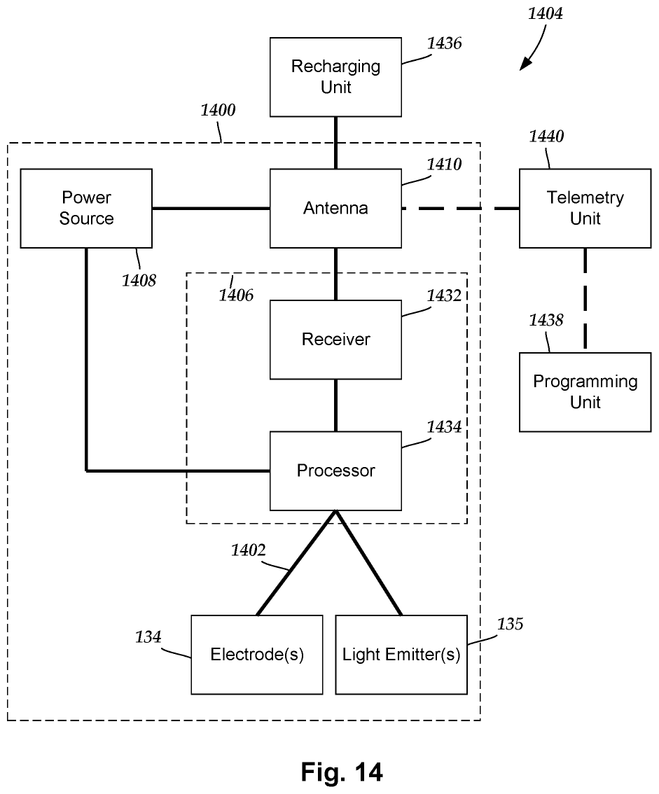

FIG. 14 is a schematic overview of one embodiment of components of a stimulation arrangement 1404 that includes an optical or optical/electrical modulation system 1400 with a lead 1402, stimulation circuitry 1406, a power source 1408, and an antenna 1410. The optical modulation system can be, for example, any of the optical or optical/electrical modulation systems described above. It will be understood that the optical or optical/electrical modulation arrangement can include more, fewer, or different components and can have a variety of different configurations including those configurations disclosed in the stimulator references cited herein.

If the power source 1408 is a rechargeable battery or chargeable capacitor, the power source may be recharged/charged using the antenna 1410, if desired. Power can be provided for recharging/charging by inductively coupling the power source 1408 through the antenna 1410 to a recharging unit 1436 external to the user. Examples of such arrangements can be found in the references identified above.

Light is emitted from the light emitter(s) 135 to provide PBM or optical modulation. In at least some embodiments, electrical current is emitted by the optional electrodes (such as electrodes 134 in FIG. 1) on the lead 1402 to stimulate nerve fibers, muscle fibers, or other body tissues near the optical or optical/electrical modulation system. The stimulation circuitry 1406 can include, among other components, a processor 1434 and a receiver 1432. The processor 1434 is included to control the timing and electrical characteristics of the optical or optical/electrical modulation system. For example, the processor 1434 can, if desired, control one or more of the timing, frequency, strength, duration, and waveform of the optical or electrical pulses. In addition, the processor 1434 can select which light emitters or optional electrodes can be used to provide stimulation, if desired.

Any processor can be used and can be as simple as an electronic device that, for example, produces pulses at a regular interval or the processor can be capable of receiving and interpreting instructions from an external programming unit 1438 that, for example, allows modification of pulse characteristics. In the illustrated embodiment, the processor 1434 is coupled to a receiver 1432 which, in turn, is coupled to the antenna 1410. This allows the processor 1434 to receive instructions from an external source to, for example, direct the pulse characteristics and the selection of light emitters or electrodes, if desired.

In at least some embodiments, the antenna 1410 is capable of receiving signals (e.g., RF signals) from an external telemetry unit 1440 that is programmed by the programming unit 1438. The programming unit 1438 can be external to, or part of, the telemetry unit 1440. The telemetry unit 1440 can be a device that is worn on the skin of the user or can be carried by the user and can have a form similar to a pager, cellular phone, or remote control, if desired. As another alternative, the telemetry unit 1440 may not be worn or carried by the user but may only be available at a home station or at a clinician's office. The programming unit 1438 can be any unit that can provide information to the telemetry unit 1440 for transmission to the optical or optical/electrical modulation system 1400. The programming unit 1438 can be part of the telemetry unit 1440 or can provide signals or information to the telemetry unit 1440 via a wireless or wired connection. One example of a suitable programming unit is a computer operated by the user or clinician to send signals to the telemetry unit 1440.

The signals sent to the processor 1434 via the antenna 1410 and the receiver 1432 can be used to modify or otherwise direct the operation of the optical or optical/electrical modulation system 1400. For example, the signals may be used to modify the pulses of the optical or optical/electrical modulation system such as modifying one or more of pulse duration, pulse frequency, pulse waveform, and pulse strength. The signals may also direct the optical or optical/electrical modulation system 1400 to cease operation, to start operation, to start charging the battery, or to stop charging the battery.

Optionally, the optical or optical/electrical modulation system 1400 may include a transmitter (not shown) coupled to the processor 1434 and the antenna 1410 for transmitting signals back to the telemetry unit 1440 or another unit capable of receiving the signals. For example, the optical or optical/electrical modulation system 1400 may transmit signals indicating whether the optical or optical/electrical modulation system 1400 is operating properly or not or indicating when the battery needs to be charged or the level of charge remaining in the battery. The processor 1434 may also be capable of transmitting information about the pulse characteristics so that a user or clinician can determine or verify the characteristics or transmitting temperature information from a temperature probe associated with the optical modulation system.

In at least some embodiments, the optical or optical/electrical modulation system 1400 can also include a thermal sensor that is disposed near the light source to monitor the light source temperature. In at least some embodiments, the optical or optical/electrical modulation system 1400 can reduce the current to the light source or turn the light source off if the thermal sensor indicates overheating or heating above a threshold temperature.

The above specification provides a description of the structure, manufacture, and use of the invention. Since many embodiments of the invention can be made without departing from the spirit and scope of the invention, the invention also resides in the claims hereinafter appended.

What is claimed as new and desired to be protected by Letters Patent of the United States is:

1. An optical lead, comprising:
a cuff body having an exterior surface and an interior surface, wherein the cuff body defines a nerve channel for receiving a portion of a nerve;
a lead body coupled, or coupleable, to the cuff body;
at least one light emitter disposed on or within the cuff body or the lead body; and
at least one reflective element disposed on, within, or beneath the interior surface of the cuff body, wherein the at least one reflective element is configured to reflect light emitted from the at least one light emitter.

2. The optical lead of claim 1, wherein the at least one reflective element comprises a reflective foil, reflective coating, or reflective particles.

3. The optical lead of claim 1, wherein the at least one reflective element comprises a reflective sheet disposed on the interior surface of the cuff body.

4. The optical lead of claim 1, wherein the at least one reflective element comprises a plurality of reflective strips disposed on the interior surface of the cuff body.

5. The optical lead of claim 1, wherein the at least one reflective element comprises a reflective layer and the cuff body comprises two polymeric layers with the reflective layer disposed therebetween.

6. The optical lead of claim 1, wherein the at least one reflective element comprises a plurality of electrodes disposed on or with the cuff body, wherein the lead body comprises a plurality of conductors coupled to the electrodes to provide electrical stimulation.

7. The optical lead of claim 1, wherein the cuff body has a spiral arrangement for self-sizing of the cuff body around a nerve.

8. The optical lead of claim 1, further comprising at least one suture tab, suture sleeve, or lead anchor configured for attaching the cuff body or lead body to tissue.

9. The optical lead of claim 1, wherein the light emitter is a light source.

10. The optical lead of claim 1, wherein the light emitter is an emission region of an optical fiber, fiber optic, or other optical waveguide.

11. A system, comprising:
the optical lead of claim 1; and
a control module coupled, or coupleable, to the optical lead and configured to direct intermittent delivery of light via the at least one light emitter.

12. An optical lead, comprising:
a cuff body having an exterior surface and an interior surface, wherein the cuff body defines a nerve channel for receiving a portion of a nerve;
a lead body coupled, or coupleable, to the cuff body and comprising a distal end portion, wherein the cuff body comprises a receptacle for removably receiving the distal end portion of the lead body, wherein the lead body and cuff body are capable of being coupled via the receptacle during a surgical procedure; and
at least one light emitter disposed on or within the cuff body or the lead body.

13. The optical lead of claim 12, wherein the receptacle is disposed on the interior surface of the cuff body.

14. The optical lead of claim 12, wherein the receptacle is disposed on the exterior surface of the cuff body.

15. The optical lead of claim 12, further comprising a fastener configured to fasten the distal end portion of the lead body to the receptacle.

16. The optical lead of claim 12, wherein at least of the at least one light emitter is disposed on or within the distal end portion of the lead body.

17. The optical lead of claim 16, wherein the lead body further comprises a redirection element disposed within the distal end portion of the lead body and configured to receive light from the at least one of the at least one light emitter and redirect the received light.

18. The optical lead of claim 16, wherein the cuff body further comprises a redirection element disposed within the receptacle of the cuff body and configured to receive light from the at least one of the at least one light emitter, when the distal end portion of the lead body is received in the receptacle, and redirect the received light into the nerve channel.

19. The optical lead of claim 16, wherein the distal end portion of the lead body and the receptacle jointly form a tongue-and-groove arrangement to maintain the coupling of the lead body and the receptacle.

20. An optical lead, comprising:
a cuff body having an exterior surface and an interior surface, wherein the cuff body defines a nerve channel for receiving a portion of a nerve;
at least one light emitter disposed on or within the cuff body;
an electronic subassembly disposed on or within the cuff body and configured to direct intermittent delivery of light via the at least one light emitter; and
an antenna disposed on or within the cuff body and coupled to the electronic subassembly and at least one light emitter for providing power from an external source.
